(19) United States
(12) Patent Application Publication
(10) Pub. No.: US 2023/0226323 A1
(43) Pub. Date: Jul. 20, 2023

(54) INTRAVENOUS CANNULA

(71) Applicant: MedSource International LLC, Chaska, MN (US)

(72) Inventors: Neeraj Gupta, Gurgaon (IN); Calvin Todd Fagley, Excelsior, MN (US); Rachel Ann Sender, Eden Prairie, MN (US)

(21) Appl. No.: 17/592,795

(22) Filed: Feb. 4, 2022

(30) Foreign Application Priority Data
Jan. 14, 2022 (IN) 202211002363

(51) Int. Cl. A61M 25/06; A61M 25/00

(52) U.S. Cl. CPC A61M 25/0625; A61M 25/0606; A61M 25/0097

(57) ABSTRACT

Intravenous cannula devices configured to prevent accidental needle prick injuries include a catheter assembly and a needle guard assembly. The catheter assembly includes a catheter hub having a coaxial recess configured to engage with a distal portion of the needle guard assembly. Intravenous cannula devices configured to prevent needle stick injuries include a safety release component at a distal end of the needle guard assembly. The safety release component includes moveable locking elements configured to fit within an annular groove defined by a proximal portion of the catheter hub upon movement of a needle through the safety release component. Proximal retraction of the needle through the safety release component causes the moveable locking elements to be displaced from the annular groove, thereby decoupling the catheter assembly from the needle guard assembly.

### TECHNICAL FIELD

The present disclosure relates to medical devices. Implementations include intravenous cannulas configured to prevent needle stick injuries. Implementations also include intravenous cannulas equipped with improved catheter coupling and release mechanisms configured to facilitate reliable, safe disengagement of a catheter assembly from a needle guard assembly after placement of a distal end of the catheter assembly within a targeted blood vessel.

### BACKGROUND

Intravenous cannulas are used to inject and/or withdraw fluids, such as medication, nutrients, or blood, directly into or out of a blood vessel of a patient. Intravenous cannulas typically include a catheter assembly and a needle insertion or protection assembly. A distal portion of a catheter tube included within the catheter assembly can be introduced into a blood vessel using a needle, after which the cannula may be secured to a patient's skin, for example, with an adhesive, which could be tape. The catheter tube included within such devices generally defines a lumen sized to accommodate insertion and retraction of a disposable hollow-bore needle therethrough. For this reason, the devices are occasionally referred to as over-the-needle cannulas.

When a distal portion of a catheter is inserted into a patient's vasculature, the disposable needle passing through the catheter is extended distally to puncture the patient's vein or artery, thereby providing an access point for the cannula to deliver or withdraw the desired fluid(s). The needle is then withdrawn, leaving the catheter assembly in place as a hub, which can be used, for example, for connections to various external hook-ups, e.g., fluid bottles. The catheter hub can also be capped for later use.

Despite their widespread and long-time use, preexisting intravenous cannulas remain problematic on multiple fronts. For example, cannulas often lack the safety features necessary to prevent catheter needles from injuring medical professionals before or after the distal tip of the needle is inserted into a patient's vasculature. Because of the high prevalence of communicable diseases among hospitalized patients in need of catheter-based treatments, the consequences of inadvertent needle pricks can be severe.

Cannulas have been designed to solve this problem by enclosing a portion of the needle within a needle guard housing moveable relative to an elongated needle tube, which may be sized to accommodate the full length of the needle before and after its deployment within a patient's vasculature. The elongated needle tube may constitute a component of a needle insertion assembly, which can be temporarily coupled to a catheter assembly, such that, after a distal portion of the catheter has been placed in a blood vessel, the needle can be withdrawn into the elongated needle tube and the needle insertion assembly disengaged from the catheter assembly. Devices configured in this manner are vulnerable to untimely separation of the catheter assembly from the needle insertion assembly, however, as they often rely entirely on sufficient but not excessive friction for coupling the two components.

Indian patent application number 3031/DEL/2014 (hereinafter referred to as the '3031 patent application) provides another example of a preexisting, but imperfect, catheter device. One problem associated with the device disclosed therein is that, when the needle is retracted from the needle cover, the needle cover does not disengage from the catheter hub, meaning more force is required to disengage the needle cover from the catheter hub. Still further, the needle disclosed in the '3031 patent application does not engage the needle cover with the catheter hub, which impedes the overall functionality of the catheter. This defect forces operators to manipulate the catheter device to extract the needle cover from the hub, which in turn may damage the targeted blood vessel and cause pain.

The present disclosure is directed to improved cannulas designed to overcome the aforementioned problems in addition to providing other technical advantages.

### SUMMARY OF THE DISCLOSURE

One object of the present disclosure is to prevent needle prick injuries that may be sustained by a medical professional before or after puncturing a vein or artery of a patient.

Embodiments of the intravenous cannula devices described herein can include a catheter assembly configured to couple with a needle guard assembly, where both components are configured to accommodate the passage of a needle therethrough. Embodiments of the catheter assembly can include a catheter hub having a proximal end and a distal end, along with a coaxial recess. “Coaxial” refers to a tube in a tube, with their axes running the same direction; the axes in a coaxial arrangement may be, but need not be, coincident.

Embodiments of cannula devices can include a flashback chamber configured to receive proximally flowing blood indicative of successfully puncturing a targeted blood vessel by the needle extending distally from the cannula.

In some embodiments, the cannula device can include a flashback chamber having a porous filter and a cover to allow air to escape and blood to flow inside the flashback chamber.

In some embodiments, the device can include a needle prick safety device or component.

In some embodiments, the needle guard assembly can include an elongated tubular member and a needle hub comprising a needle holder disposed inside the elongated tubular member. A distal end of the needle holder can be connected to the needle configured to puncture a targeted blood vessel within a patient. Embodiments can also include a safety release component fixed or coupled to a distal end of the elongated tubular member. The safety release component can be configured to releasably couple with the catheter hub, thereby coupling the catheter assembly with the needle guard assembly. The safety release component can include one or more locking elements at or near its distal end, which can be configured to project and/or move radially outwardly when a needle is inserted within the safety release component. Outward projection and/or movement of the locking elements may cause them to fit within, and thus engage with, an annular groove defined by a proximal portion of the catheter hub, thereby forming a locking engagement and/or a tight fit relationship between the needle guard assembly and the catheter assembly when the needle is passed through the safety release component to puncture a patient's blood vessel. When the needle is retracted through the catheter assembly and the safety release component after puncturing the patient's blood vessel, the locking elements retract, and/or can be displaced, from the annular groove of the catheter hub, thereby disengaging the safety release component and catheter hub. This disengagement allows separation of the catheter assembly from the needle guard assembly, but only when the needle is enclosed within the safety release component and needle guard assembly. In this manner, embodiments of the cannula devices disclosed herein can shield the distal tip of the needle upon its withdrawal from a patient to prevent inadvertent needle stick injuries.

In some embodiments, the safety release component can define a groove on an outer surface to accommodate the one or more locking elements.

In some embodiments, the one or more locking elements can be solid and spherical. The locking element(s) can also be made of a rigid or substantially rigid material, such as a metal, e.g., stainless steel and/or Nitinol and/or etc.

In some embodiments, each groove of the safety release component can have a diameter greater than or equal to a diameter of the locking element(s).

In some embodiments, the proximal end of the safety release component can have a circular base portion defining a central bore and fixed with the distal end of the elongated tubular member.

In some embodiments, the safety release component can include a tubular portion extending from the circular base portion and defining an axial bore configured to accommodate the passage of a needle to and from a targeted insertion site.

In some embodiments, the groove defined by the outer surface of the tubular portion of the safety release component can extend toward the axial bore of the safety release component.

In some embodiments, the solid spherical elements of the safety release component can be configured to extend outwardly to engage with the annular groove of the catheter hub, thereby forming the locking engagement and tight fit relationship between the elongated tubular member and the catheter assembly when a needle is passed through the safety release component pursuant to puncturing a blood vessel of a patient.

In some embodiments, when the needle is retracted proximally through the safety release component after puncturing the blood vessel of a patient, the locking elements of the safety release component decouple from the annular groove defined by the catheter hub, thereby disengaging the safety release component (and needle guard assembly) from the catheter assembly. Disengagement of the safety release component from the catheter assembly allows separation of the catheter assembly from the needle guard assembly, but only after the distal tip of the needle has been fully enclosed within the safety release component.

In some embodiments, the safety release component included in the needle guard assembly can include a safety clip. The safety clip may bias outwardly to engage at least one interlocking flange defined in a body portion of the needle guard assembly. Outward biasing of the safety clip can allow a needle member to extend through the needle guard assembly and catheter hub. Upon withdrawal of the needle member from the proximal end of the catheter hub, the safety clip may disengage from the interlocking flange and enclose a tip portion of the needle member within the safety clip, thereby reducing the likelihood of needle prick injuries upon withdrawal of the needle member from the catheter hub.

In some embodiments, the safety clip may comprise a bracket defining an opening for receiving the needle member. A first resilient arm may extend from one end of the bracket and may have a connecting portion for engaging with the at least one portion with the at least one interlocking flange of the body portion and needle member. The first resilient arm can comprise a first section and a section. A second resilient arm can extend from an opposing end of the bracket and can further comprise a connecting portion for engaging with the at least one interlocking flange of the body portion and the needle member. The second resilient arm can also include a first section and a second section, and the dimensions of the first section can be larger than the second section. The connecting portion of each of the first and second resilient arms can be configured to engage with the interlocking flange and a body portion of the needle member when the needle member is extending through the catheter hub en route to puncturing a patient's blood vessel. The connecting portion can disengage from the interlocking flange and body of the needle member when the needle member is withdrawn from the proximal end of the catheter hub.

In some embodiments, the connecting portion of each of the first and second resilient arms includes a curved protrusion at one end. The curved protrusion can be configured to engage with the at least one interlocking flange of the body portion and a curved lip segment. The curved lip segment can extend inwardly toward the bracket member and can be configured to engage with the needle member. The curved lip can be configured to enclose the tip portion of the needle member within the safety clip when the needle member is withdrawn from the proximal end of the catheter hub.

In some embodiments, the curved protrusion includes a projection extending towards the bracket. The projection, together with the second section of the first and second resilient arms, can define a seat portion, which can be configured to receive and seat the tip portion of the needle member, thereby preventing misalignment of the needle member during withdrawal from catheter hub.

In some embodiments, the first resilient arm can be longer than the second resilient arm.

In some embodiments, at least one of the bracket, the first resilient arm, and/or the second resilient arm can be defined with at least one rib member for reinforcement.

In some embodiments, the rib member can be disposed at a second section of the first and second resilient arms to reinforce the first and second resilient arms.

In some embodiments, each of the at least one interlocking flange of the body portion extends radially inwardly for ensuring engagement with the safety clip in its biased configuration, so that the safety clip is retained within the needle guard assembly.

In some embodiments, the needle member can include a protuberance proximal to its distal end. The protuberance can be configured to engage with the bracket during withdrawal of the needle member from the proximal end of the catheter tube, thereby preventing release of the needle member from the needle guard assembly.

Other features and aspects of this disclosure will be apparent from the following description and the accompanying drawings.

### BRIEF DESCRIPTION OF THE DRAWINGS

FIG. 1 is a perspective view of an example of an intravenous cannula device in accordance with embodiments of the present disclosure.

FIG. 2A is a cross-sectional view of the intravenous cannula device of FIG. 1, showing the catheter assembly coupled with the needle guard assembly.

FIG. 2B is a cross-sectional view of the intravenous cannula device of FIG. 1, showing the catheter assembly separated from the needle guard assembly.

FIG. 3A is a cross-sectional view of an example of a safety release component coupled with a needle guard assembly in accordance with embodiments of the present disclosure.

FIG. 3B is a perspective view of the safety release component shown in FIG. 3A.

FIG. 3C is another cross-sectional view of the safety release component shown in FIG. 3A.

FIG. 3D is a cross-sectional view of an example of a configuration of the safety release component when a distal tip of a needle is enclosed within the needle safety component.

FIG. 4A is a cross-sectional view of an example of another intravenous cannula device in accordance with embodiments of the present disclosure.

FIG. 4B is a magnified cross-sectional view of a portion of the intravenous cannula device of FIG. 4A, showing the safety release component coupled with the needle guard assembly in accordance with embodiments of the present disclosure.

FIG. 5 is a cross-sectional view of an example of a needle guard assembly in accordance with embodiments of the present disclosure.

FIGS. 6A, 6B, 6C, 6D, and 6E are progressive snapshots of a portion of the needle guard assembly shown in FIG. 5 as the configuration of the needle guard assembly moves from an injection state to a shielded state.

FIG. 7 is a cross-sectional view of an example of a duckbill component configured to releasably couple a catheter assembly to a needle guard assembly in accordance with embodiments of the present disclosure.

FIG. 8 is a cross-sectional view of another example of an intravenous cannula device in accordance with embodiments of the present disclosure.

FIG. 9 is a perspective view of a safety clip disposed within a needle guard assembly of the cannula device shown in FIG. 8 according to embodiments of the present disclosure.

FIG. 10 is a cross-sectional view of the cannula device shown in FIG. 8 when the needle member is withdrawn from the catheter hub, according to embodiments of the present disclosure.

### DETAILED DESCRIPTION

Provided herein are non-limiting embodiments of the present disclosure. References to specific embodiments and features are detailed throughout this disclosure, and examples are illustrated in the accompanying drawings. Reference numbers are included in the drawings to refer to the same or corresponding parts. References to various elements described herein are made collectively or individually when there may be more than one element of the same type; however, such references are merely exemplary in nature. Any reference to elements in singular form may also be construed to relate to plural form and vice-versa without limiting the scope of the disclosure to the exact number or type of such elements, unless set forth explicitly in the text.

As used herein, the term “proximal end” may refer to an end closer to the operator of a disclosed device. The term “distal end” as used herein may refer to an end opposite the “proximal end,” which may be closer to the patient being treated by a disclosed device. Accordingly, the terms “distal” or “distal end” and “proximal” or “proximal end” may refer to directions or ends which are respectively further from and closer to the operator inserting a catheter into the body of a patient.

As used herein, the terms “operator” and “user” may be used interchangeably and may include, but are not limited to, medical professionals and personnel, such as nurses or para-medical staff who may work under the direction and supervision of doctors, physicians, and/or surgeons, who may also be considered users or operators according to the embodiments described herein.

The terms “connected” or “fixedly connected” as used in the present disclosure may refer to components that may be attached to each other in a fixed manner, which may be permanent in the sense that disconnection would require specialized tools and/or excessive physical force. “Releasably connected” or “coupled” may refer to components that may be temporarily connected and disconnected via one or more device features. The term “slidably connected” may refer to components assembled together in such a manner that any one or more of the components can slide relative to the other(s) during device employment. The contact surfaces of the components may enable such sliding. The term “disposed” used herein may mean that a component or element of a device may be connected to another element such that a workable assembly is formed without hindering the functionality of each individual element. The term “comprising” means that a given device or components thereof may include additional components apart from the components explicitly identified herein.

This disclosure includes numeric terms and phrases such as “one or more,” “at least,” “a,” and “an.” The specific numbers associated with such terms should not be construed as limiting.

Terms defining shapes, e.g., “convex,” “frustoconical,” “flat,” “substantially flat,” “cylindrical,” “tubular,” “extended,” “circular,” “converging,” “diverging,” “tapered,” or “expanding” should also not be construed as limiting. A person of ordinary skill would recognize that these shapes allow for some variation, e.g., a “circular” shape, whether or not modified by a term such as “generally” or “substantially,” need not meet the theoretical definition of “circular” to be circular within the meaning of the term as used herein. Other shapes may be possible in certain embodiments. As such, a person of ordinary skill in the art may develop other shapes or shape combinations that preserve the workability of a disclosed device. Any of such alterations may still be encompassed within the present disclosure without departing from the invention.

The terminology used in the present disclosure includes the words specifically mentioned, derivatives thereof, and words of similar import. The embodiments illustrated below are not intended to be exhaustive or to limit the invention to the precise form disclosed. These embodiments are chosen and described to best explain the principles of the invention and its application, its practical use, and to enable others skilled in the art to best utilize and develop the invention.

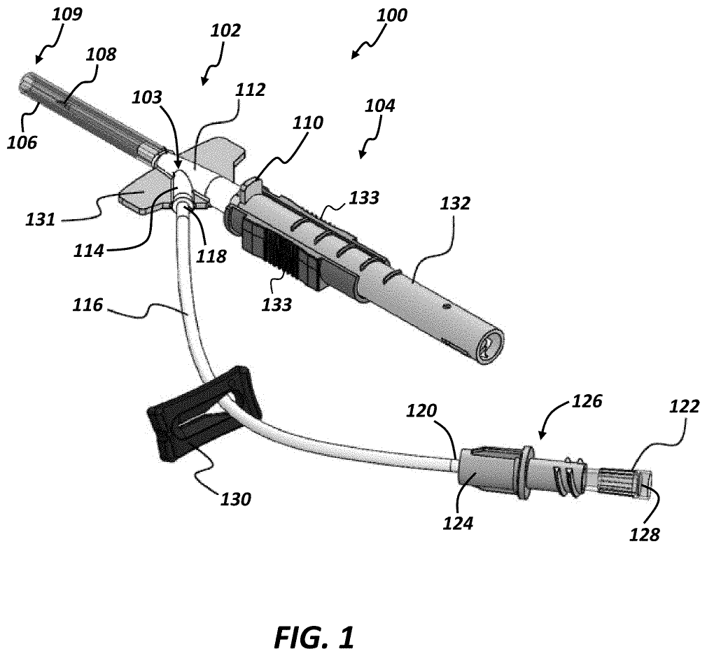

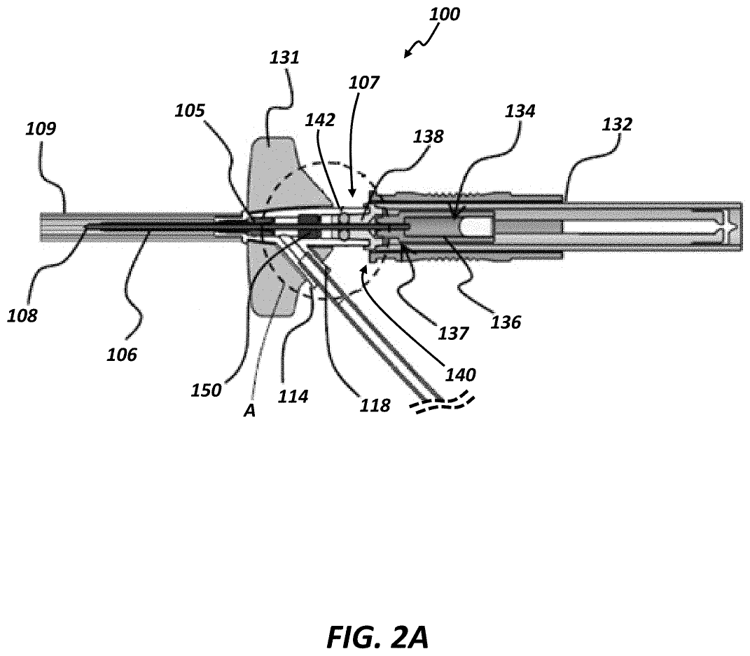

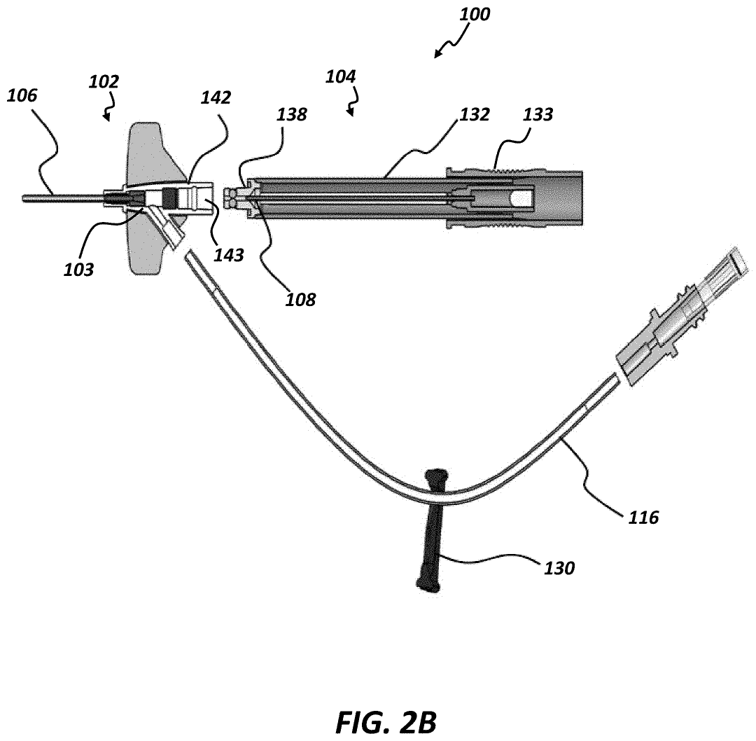

FIGS. 1 and 2 illustrate a perspective view and a sectional view of a cannula 100, respectively, according to embodiments of the present disclosure. The illustrated cannula 100 is a medical device that can be used to administer a fluid medication via intravenous therapy and/or remove bodily fluids, e.g., blood, from a patient for subsequent analysis. The particular type of cannula device disclosed herein may vary, as can the associated tasks performed therewith. In the illustrated figures, the cannula 100 is an intravenous cannula device. The term “intravenous cannula” is used interchangeably with “cannula” herein for brevity.

The cannula 100 illustrated in FIGS. 1 and 2 includes a catheter assembly 102 configured to reversibly couple with a needle hub, chamber, or guard assembly 104. The snapshot shown in FIG. 1 depicts the catheter assembly 102 coupled with the needle guard assembly 104.

The catheter assembly 102 includes a body member 103 surrounding a catheter hub 105, which can be fixed or coupled with a catheter tube 106. A needle 108 is shown within the catheter tube 106, which can be made of a flexible or soft material, non-limiting examples of which may include a plastic or polymer composition. In embodiments, other components of the catheter assembly 102, such as the body member 103 and catheter hub 105, can be made of a biocompatible material, which can be substantially rigid.

The cannula 100 is configured such that the catheter assembly 102 can be decoupled from the needle guard assembly 104 after the needle 108 pierces a targeted blood vessel and is retracted proximally through catheter assembly 102, thereby leaving at least a portion of the distal portion 109 of the catheter assembly 102 within the blood vessel to facilitate the delivery and/or withdrawal of various medications and/or bodily fluids. Embodiments of the cannula 100 also can be configured to prevent inadvertent needle prick injuries by fully enclosing the needle 108, including its distal tip, within the needle guard assembly 104 after piercing a targeted blood vessel.

The catheter tube 106 defines an elongated, longitudinal bore through which the needle 108 can slide. The catheter tube 106 can be fixed with the catheter assembly 102 by a number of methods including, but not limited to, press fitting, adhesive bonding, or any other suitable method, which may be applied to the catheter hub 105, specifically. In some examples, the catheter tube 106 may be formed integrally with the catheter assembly 102. For gripping and manipulating the cannula 100 to reposition the needle tube 106 during insertion and retraction of the needle 108, a thumb grip 110 can also be included.

In the illustrated embodiment, the body member 103 is a Y-type body member, but this example is not limiting, as other shapes and sizes of the body member 103 may be compatible with one or more embodiments disclosed herein. The illustrated Y-type body member 103 has a cylindrical body portion 112 along the catheter hub 105 and a tangential portion 114 connected to the cylindrical body portion 112. Both the cylindrical body portion 112 and the tangential portion 114 can be integrally formed or manufactured. In another embodiment, the tangential portion 114 may be releasably connected to the cylindrical body portion 112. The body member 103 may be made of a medical grade plastic known in the art. The tangential portion 114 may be connected to a tube 116 for administering the fluids as described above.

One end 118 of the tube 116 may be connected to the tangential portion 114 of the body member 118 and a second end 120 of the tube 116 may be connected to a flashback chamber 122. The flashback chamber 122 may include a female luer 124, a flow control hub 126 and a filter 128. Blood flow into the flashback chamber 122 from a patient confirms successful puncturing of a vein or artery by the needle 108.

The tube 116 may be further provided with a slide clamp 130 for arranging or connecting the tube 116 to a stand or support column. One or more wing members 131 can also be coupled or integrally formed with the body member 103. The wing member(s) 131 may aid in connecting or affixing the catheter assembly 102 with a patient's clothing or body part, e.g., a hand or arm, after puncturing a targeted blood vessel. Any luer lock member of standard size or having the ISO standards which conforms with the catheter assembly 102 can be used in various embodiments. For example, the ISO standards ISO-80369-20 and ISO-80369-7 can be used for a luer lock member having a 6% universal taper. In additional embodiments, the luer lock member 124 may have other configurations or shapes, such as a luer slip.

The needle guard assembly 104 can include an elongated tubular member 132 surrounded at least in part by a gripping member or surface 133. The elongated tubular member 132 may contain a needle hub 134 comprising a needle holder 136. The needle holder 136 is disposed inside the elongated tubular member 132, where a distal portion 137 of the needle holder 136 can be connected to the needle 108. The elongated tubular member 132 may be sized to accommodate the entire length of the needle 108, such that retraction of the needle 108 into the elongated tubular member 132 encloses and shields the distal tip of the needle 108. The shape of the elongated tubular member 132 may vary, and may be cylindrical, rectangular, or any other suitable configuration.

The needle guard assembly 104 also includes a safety release component 138 fixed to a distal end 140 of the elongated tubular member 132. The safety release component 138 can be releasably coupled to the body member 103 of the catheter assembly 102, such that after coupling the catheter assembly 102 to the needle guard assembly 104, the safety release component 138 is positioned between the proximal end 107 of the body member 103 and the distal end 140 of the elongated tubular member 132.

FIG. 2B provides a snapshot cross-sectional view of the cannula 100 when the catheter assembly 102 is not coupled with the needle guard assembly 104. As shown, the safety release component 138 is separated from a body member 103 of the catheter assembly 102 and the needle 108 is enclosed within the needle guard assembly 104.

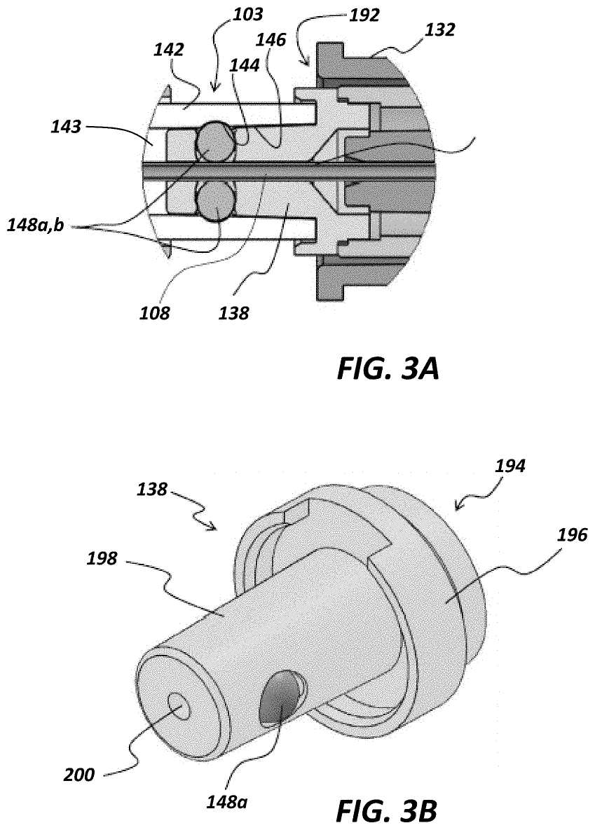

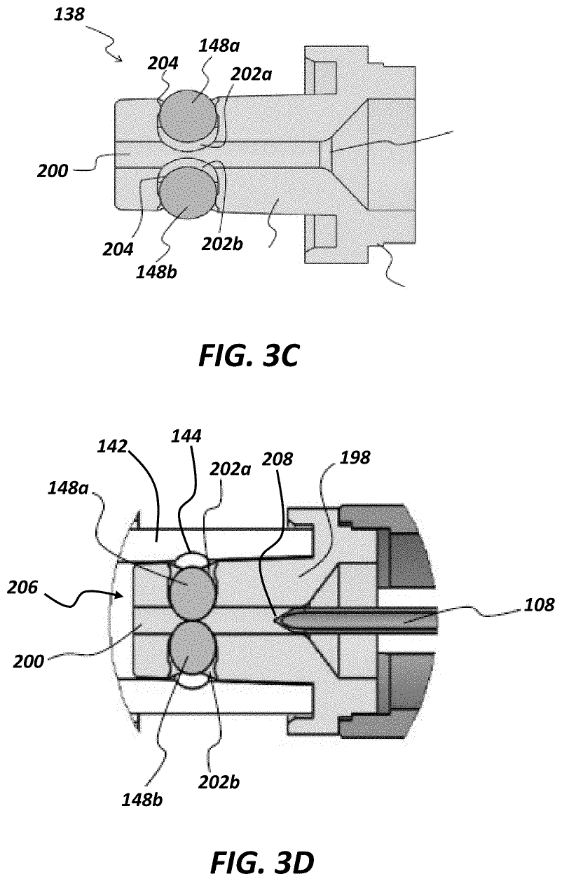

As shown together with FIG. 3A, to facilitate coupling of the catheter assembly 102 with the needle guard assembly 104, the body member 103 includes a proximal cylindrical portion 142 defining a coaxial bore 143 and an annular groove 144 at its inner surface 146. The annular groove 144 of the proximal cylindrical portion 142 is complementary to, and thus configured to engage with, one or more locking elements 148a,b of the safety release component 138. The locking elements 148a,b can be inserted within the annular groove 144 only when the safety release component 138 is inserted within the coaxial bore 143 of the proximal cylindrical portion 142 and the needle 108 has been inserted within the safety release component 138. By tightly mating with the body member 103 upon insertion of the needle 108 therethrough, the safety release component 138 increases the catheter separation force, thereby reducing the likelihood of the catheter assembly 102 uncoupling from the needle guard assembly 104 while the needle 108 remains positioned within the catheter assembly 102, in whole or in part. Likewise, upon retraction of the needle 108 proximally through the catheter assembly 102, the safety release component 138, and into the needle guard assembly 104, the catheter release force is decreased significantly, such that detachment of the needle guard assembly 104 from the catheter assembly 102 requires a relatively small amount of force. This reduced catheter release force enables easy removal of the needle guard assembly 104 by an operator, which lessens the likelihood of perturbing the placement of the catheter assembly 102 within the patient. In the illustrated embodiment, the two locking elements 148a,b are solid and spherical, but embodiments are not limited to solid, spherical locking elements. In lieu of grooves, notches, a ridge, or protrusions may be used.

After passing through the safety release mechanism 138 and the proximal cylindrical portion 142, the needle 108 extends distally through a rubber valve member 150 disposed within the body member 103, continuing through the catheter hub 105 and the catheter tube 106 en route to puncturing a targeted blood vessel.

In embodiments, a distal portion of the needle guard assembly 104, e.g., the safety release component 138, and/or a proximal portion of the catheter assembly 102, e.g., the proximal cylindrical portion 142, can include one or more radial seals configured to prevent entry and escape of fluids passing through or around the cannula 100.

The cannula 100 can be thus equipped with a needle prick prevention mechanism configured to enclose the distal tip of the needle 108 within the needle guard assembly 104 before and after insertion of the needle 108 within a patient. Safety release component 138 is an example of a component of a needle prick safety device implemented in accordance with embodiments disclosed herein. The safety release component 138 can include two moveable locking elements 148a,b configured to control the coupling of the catheter assembly 102 to the needle guard assembly 104 based on whether the needle 108 is present within either or both components.

As noted above, FIG. 3A shows a close-up sectional view of the safety release component 138 and the surrounding features in an engaged, locked state with the proximal cylindrical portion 142 of the body member 103. As shown, the locking elements 148a,b are each engaged or locked with a portion of the annular groove 144 defined by an inner surface 146 of the proximal cylindrical portion 142 of the body member 103. In this engaged state, the safety release component 138 is fixed to a distal end 192 of the elongated tubular member 132 and releasably connected to the proximal cylindrical portion 142 of the body member 103.

FIG. 3B is a perspective view of the safety release component 138 according to embodiments disclosed herein. The safety release component 138 comprises a first, proximal end 194 having a circular base portion 196, which can be fixed to the distal end 192 of the elongated tubular member 132. In some examples, the circular base portion 196 can be press-fitted to the elongated tubular member 132, thereby unifying the two components. The safety release component 138 can also comprise a tubular portion 198 defining an axial bore 200 sized to accommodate passage of the needle 108 therethrough.

FIGS. 3C and 3D show the safety release component 138 in a disengaged or unlocked state relative to the body member 103 at a snapshot in time during which the needle 108 is either being retracted proximally or extended distally, such that the needle tip is momentarily positioned within the safety release component 138, where the tip is fully enclosed. The needle tip 208 is not yet fully withdrawn into the needle guard assembly 104, but is located in a safe, unexposed area within the safety release component 138 such that upon complete separation of the safety release component 138 (and needle guard assembly 104) from the body member 103, the needle tip 208 may not cause accidental prick injuries. In the disengaged or unlocked state, the safety release component 138 may not be coupled with the body member 103 or may at least not be locked with the body member 103 but in the process of being separated therefrom, such that the safety release component 138 and needle guard assembly 104 to which it is attached may be readily separated in unison from the catheter assembly 102 by applying a small amount of tension force. As shown, the safety release component 138 can include one or more grooves 202a,b defined by the outer surface 204 of the tubular portion 198. Each of the grooves 202a,b is sized to accommodate one of locking elements 148a,b. The solid, spherical locking elements can be in the form of stainless steel balls, but the disclosed embodiments are not limited thereto. The locking elements may be any suitable shape, including but not limited to spherical, generally spherical, prolate spheroid, cylindrical (with or without tabs or other elements to keep the locking elements in the grooves 202a,b), conical, etc. The locking elements may be disconnected from and/or floating within the grooves, and/or they may be connected to or integral with the grooves, such as a shape tethered to a groove.

The diameter of each groove 202a,b can be substantially the same, or slightly greater, as the diameter of each corresponding locking element 148a,b. The size of each groove 202a,b facilitates smooth movement of the locking elements 148a,b radially outward, away from the axial bore 200, when the needle 108 is passed through the bore 200 pursuant to puncturing a vein or artery, and radially inward after the subsequent withdrawal of the needle 108 from the patient and proximally through the safety release component 138.

The locking elements 148a,b of the safety release component 138 are configured to engage with the annular groove 144 of the body member 103, thereby forming a locking, tight-fit engagement between the elongated tubular member 132 of the needle guard assembly 104 and the catheter assembly 102 when the needle 108 is passed through the safety release component 138 for puncturing a vein or artery of a patient. The needle 108 thus displaces the locking elements 148a,b away from the axial bore 200 and into the inner groove 144 to lock the catheter assembly 102 to the needle guard assembly 104. FIG. 3D shows that, after withdrawing the needle 108 from the patient, through the catheter assembly 102, and proximal to a distal end 206 of the needle guard assembly 104, a distal tip 208 of the needle 108 can be briefly nested within the safety release component 138 (and shortly thereafter, the needle guard assembly 104), at which time the locking engagement between the body member 103 and needle guard assembly 104 can be released. The disengagement occurs because the locking elements 148a,b are able to be displaced inwardly, after retraction of the needle, from the annular groove 144 of the proximal cylindrical portion 142 of the body member 103, thereby disengaging the locking engagement between the elongated tubular member 132 and the catheter assembly 102. Separation of the safety release component 138 from the catheter assembly 102 after nesting the needle tip 208 within the safety release component 138 (and for a greater length of time, within the needle guard assembly 104) allows the needle 108 to be withdrawn from a patient and removed from the catheter assembly 102 only when its distal tip 208 is concealed, thereby reducing the likelihood of needle prick injuries.

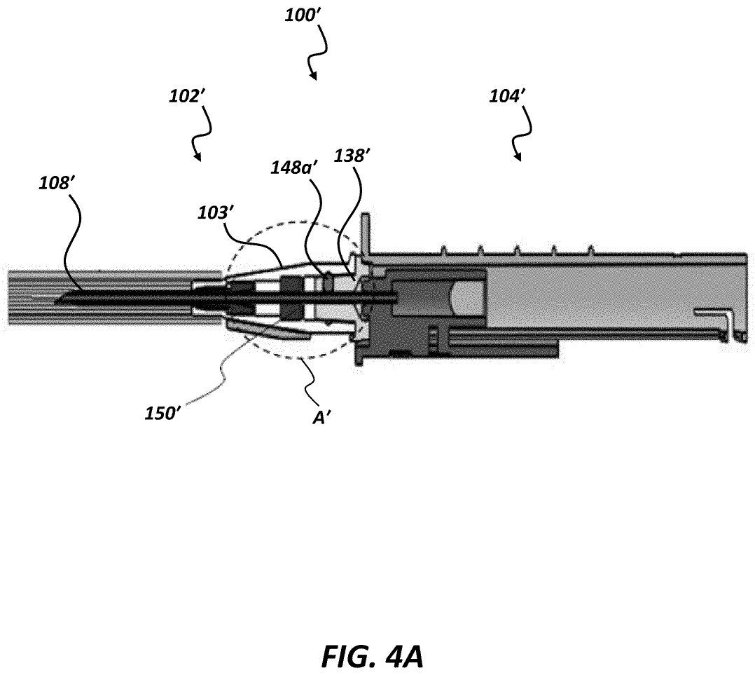

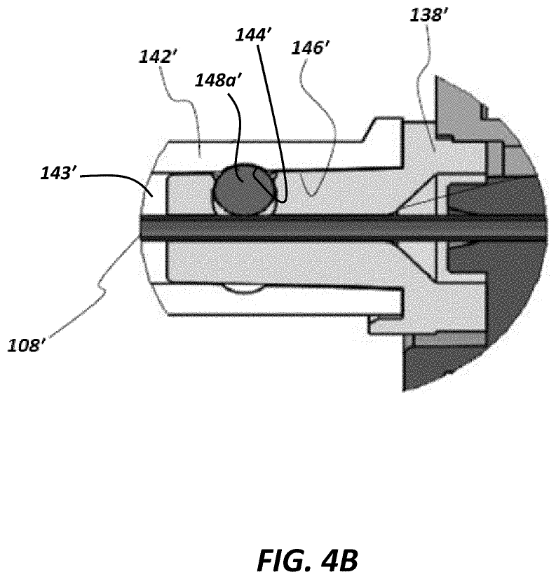

While FIGS. 2 and 3A-3D depict an embodiment of the cannula 100 having two moveable locking elements 148a,b, additional examples may include greater or less than two locking elements. As shown in FIG. 4A, an alternative embodiment of the cannula 100′ can feature a safety release component 138′ having only one locking element 148a′. FIG. 4B provides a close-up view of the cannula 100′ at A′. Like cannula 100, cannula 100′ includes a body member 103′ having a proximal cylindrical portion 142′ that defines an annular groove 144′ at its inner surface 146′. The annular groove 144′ of the proximal cylindrical portion 142′ is complementary to, and thus configured to engage with, the moveable locking element 148a′ of the safety release component 138′. The locking element 148a′ can be inserted within the annular groove 144′ only when the safety release component 138′ is inserted within the coaxial bore 143′ of the proximal cylindrical portion 142′ and the needle 108′ has been inserted within the safety release component 138′.

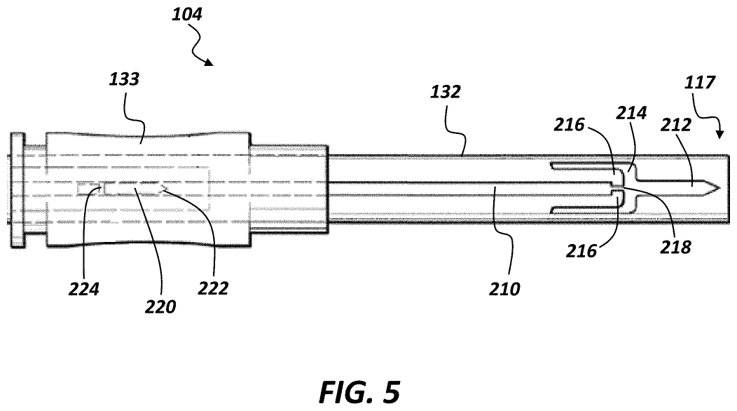

Embodiments of the cannulas described herein can also include a mechanism for ensuring safe, reliable locking and unlocking of the needle guard assembly 104 (or 104′) in a manner that further reduces the likelihood of needle prick injuries. FIG. 5 is a cross-sectional view of an example of a portion of the needle guard assembly 104 configured in this manner. As shown, the interior of the needle guard assembly 104 can include a longitudinal notch 210 defining a proximal receiving area 212. The notch 210 may further define a U-shaped slot detent cutout or notch 214, which defines a pair of fingers 216. Together, the fingers 216 define a narrow slot 218. The fingers 216 can be configured to be urged away from each other to widen the slot 218 pursuant to locking the needle guard assembly 104.

As illustrated together with FIG. 2A, the needle guard assembly 104 may be configured to have an injection position, in which the gripping member or surface 133 has been extended distally such that the needle 108 extends beyond the distal end 140 of the needle guard assembly 104, and a shielded position in which the distal tip of the needle 108 is positioned proximal to the distal end 140, nestled within the needle guard assembly 104. To move the needle hub 134 from the injection position to the shielded position, the guard assembly gripping member or surface 133 can be moved proximally, such that a projection or rib 220 also included within the needle guard assembly 104 moves proximally through the longitudinal notch 210.

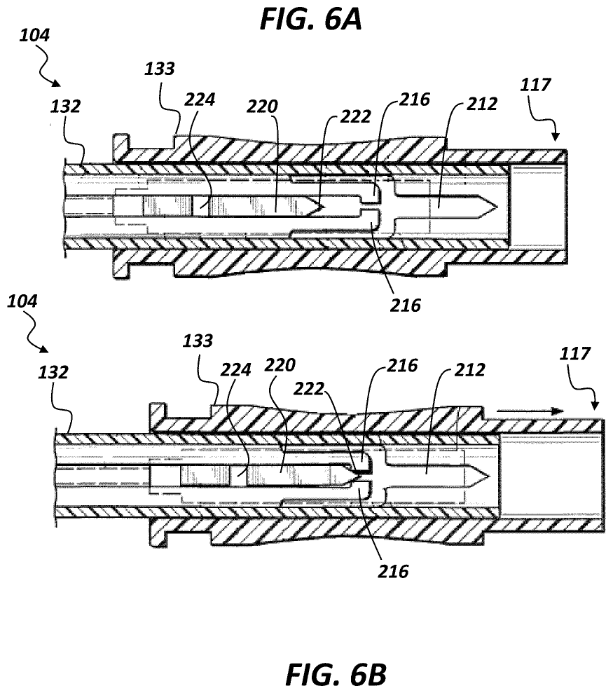

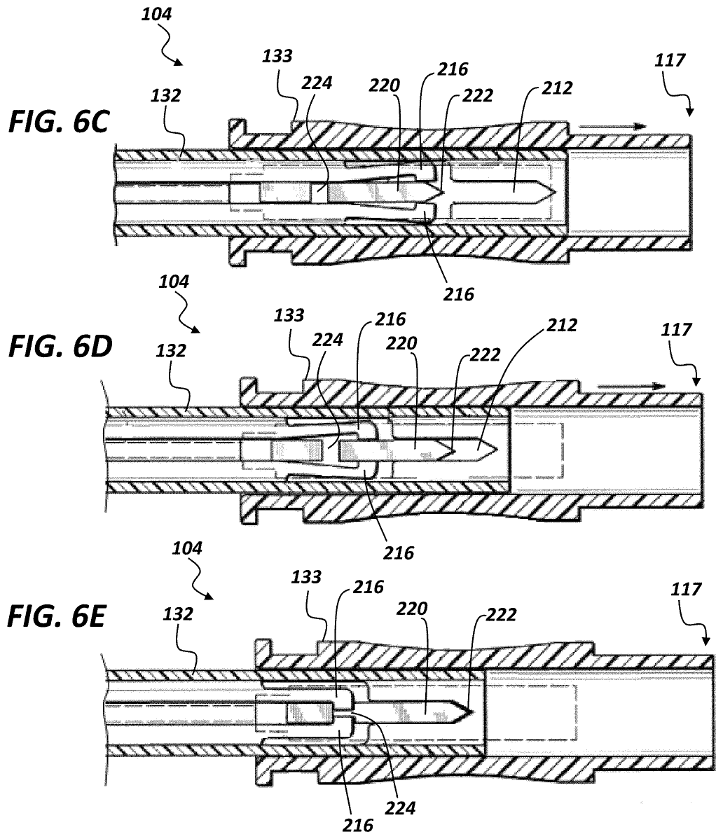

The proximal end of the rib 220 can define a ramp or camming surface 222. In a resting state, i.e., before passage of the rib 220 therethrough, the slot 218 defined by the fingers 216 can be more narrow than the width or thickness of the rib 220. As the needle hub 134 is moved proximally toward the shielded position, the camming surface 222 of the rib 220 engages the fingers 216 and urges them apart, thereby widening the slot 218 to allow passage of the rib 220 therethrough. Proximal movement of the rib 220 through the longitudinal notch 210 and beyond the fingers 216 is shown progressively in FIGS. 6A, 6B, 6C, and 6D.

As the fingers 216 are urged apart, the needle hub 134 enters a lock actuation stage in which the force generated by the camming action of the camming surface 222 against the fingers 216 exerts increased resistance to movement of the needle hub 134. Entry into the lock actuation stage is shown in FIG. 6B and passage through the lock actuation stage is shown in FIG. 6C.

After the fingers 216 are urged apart to the extent necessary for the rib 220 to enter the slot 218, the total force acting against the movement of the rib 220 is exerted by the sliding action of the fingers 216 against the rib 220. This force decreases with continued proximal movement of the needle hub 134, and thus the rib 220, toward the shielded state, which is illustrated in FIGS. 6D and 6E.

Continued proximal movement of the needle hub 134 moves the rib gap 224 beyond the proximal end of the fingers 216, thereby allowing the fingers 216 to snap back to their original non-flexed state in which the ends of the fingers 216 settle within the rib gap 224. This configuration defines the locked, shielded state in which the needle 108 cannot be pushed distally without a high external force applied in the distal direction. To prevent additional proximal movement of the needle hub 134 relative to the elongated tube member 132, the camming surface 222 can extend to and contact the proximal end of the receiving area 212.

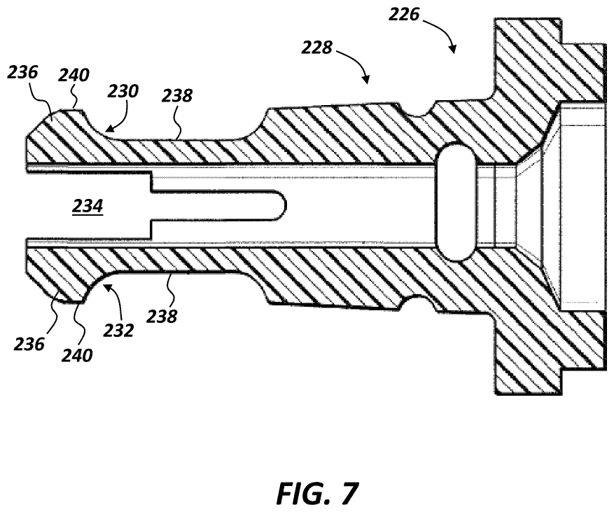

In some examples, the cannula 100 can additionally or alternatively include a duckbill release mechanism at the distal end 140 of the needle guard assembly 104, an example of which is shown in FIG. 7. As shown, the duckbill release mechanism can include a pair of cooperating members, e.g., arms, each extending distally from the distal end 140 of the needle guard assembly 104. The arms can be included in lieu of the moveable locking elements 148a,b of the safety release component 138 disclosed herein. Like the safety release component 138, the arms can be sized to fit within the proximal cylindrical portion 142 of the body member 103. The forces exerted upon entry into the lock actuation stage described in connection with FIGS. 6A-6D can be exerted while the duckbill release mechanism secures the needle guard assembly 104 to the catheter assembly 102. The safety release component 138 and duckbill release mechanism may be included in separate embodiments, such that the two release mechanisms are not included in the same device.

The cooperating arms can define a passageway therebetween configured to slidably receive the needle 108. One or both of the members may have a holding portion, such as a radially outward-protruding detent and/or radially inward extending recess configured for coupling with a complementary feature of the body member 103, which may be defined by the inner surface 146 of the proximal cylindrical portion 142. Inclusion of the duckbill mechanism can ensure a strong coupling of the catheter assembly 102 with the needle guard assembly 104 when the needle 108 has been inserted therethrough. The duckbill mechanism may facilitate easy uncoupling of the catheter assembly 102 from the needle guard assembly 104 when the needle 108 is not present.

With reference again to FIG. 7, an example of a duckbill mechanism included in some embodiments can include a distal cap 226 comprised of a nose 228 configured to be removably coupled with the proximal cylindrical portion 142 of the body member 103, such that the distal cap 226 abuts the inner surface 146 of the proximal cylindrical portion 142. A pair of distally extending arms 230, 232 defining a split cylinder are also sized to fit within the coaxial bore 143 of the body member 103. The arms 230, 232 can flex radially toward each other upon receiving a compression force, but in the uncompressed, relaxed state, they can define a passageway 234 therebetween configured to slidably receive the needle 108. Alternatively, the arms 230, 232 can be biased inwardly toward one another and pushed into a parallel configuration when the needle is between them. The end of one or each arm 230, 232 can include a holding portion in the form of a protrusion or detent 236. The inclusion of one or more detents 236 defines one or more recesses 238 proximal thereto. The outer periphery 240 of the detents 236 can define an annular ring having a diameter that is at least slightly larger than the inner diameter of the proximal cylindrical portion 142 of the body member 103. In some examples, the detents 238 may be distally chamfered.

When the needle 108 is present in the passageway 234 between the arms 230, 232, compression of the arms 230, 232 is impeded such that uncoupling of the distal cap 226 from the coaxial bore 143 of the body member 103 requires considerable force, thereby reducing the likelihood of the catheter assembly 102 separating from the needle guard assembly 104 when the needle 108 remains within the catheter assembly 102. Alternatively, when the arms are biased toward one another, retraction of the needle results in the arms moving toward one another, away from the catheter hub's interior walls.

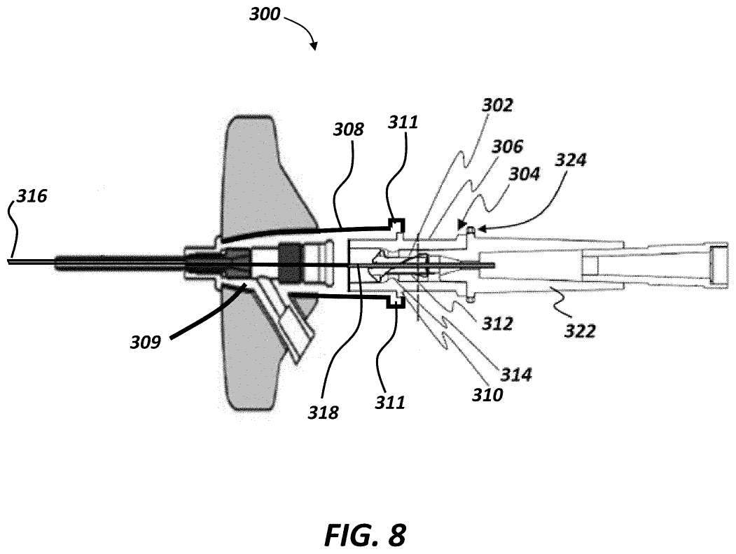

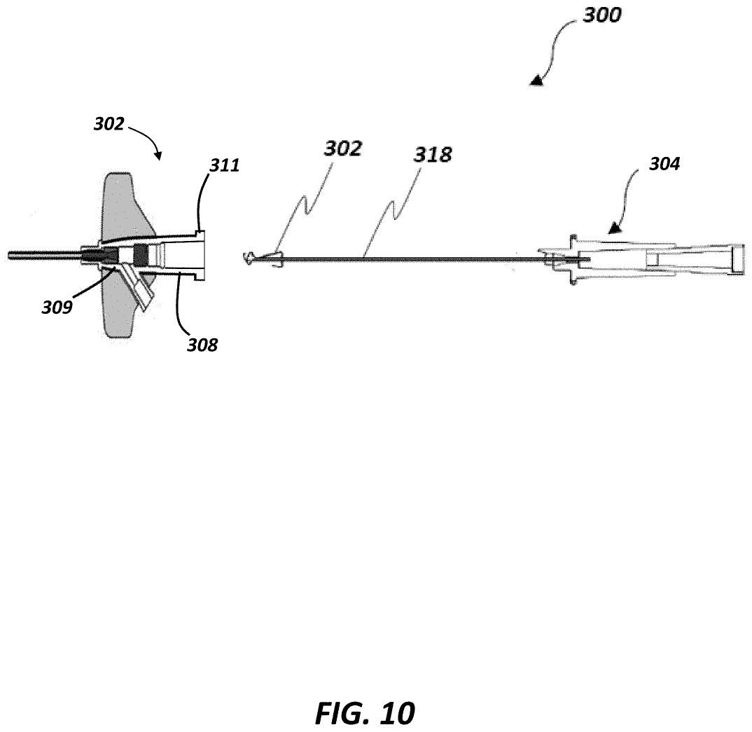

Embodiments of the disclosed devices may include additional and/or alternative structures. As shown in FIGS. 8-10, for example, a cannula 300 can include a safety clip 302 in addition to or instead of the aforementioned ball release mechanism to prevent accidental needle pricks. The cannula 300 can include a needle guard assembly 304 having a body portion 306 connected to a body member 308 coupled with a catheter hub 309 of the catheter assembly such that a projection 310 on the body portion 306 engages with a recess 311 in the body member 308. The body portion 306 of the needle guard assembly 304 defines an inner bore 312 configured for receiving the safety clip 302. The safety clip 302 can be positioned within the body portion 306 such that the safety clip 302 engages with at least one interlocking flange 314 defined in the body portion 306. In its resting, biased configuration, the safety clip 302 can allow a needle to extend through the body portion 306 and the body member 308. Upon withdrawal of the needle from the proximal end of the body member 308, the safety clip 302 can disengage from the interlocking flange 314 and enclose a tip portion 316 of the needle 318 within the body member 308, thereby preventing needle prick injuries during withdrawal of the needle 318 from a patient.

A needle hub 322 can be tight-fit relationship with the body portion 306 and fixedly connected to the needle 318. As such, when the needle 318 is withdrawn from the needle hub 322, the needle hub 322 disengages from the body portion 306 and is withdrawn from the body member 308, and the needle hub 322 disengages from the body portion 306. Due to the construction of the safety clip 302 within the body portion 306, the safety clip 302 also disengages from the body portion 306 and is withdrawn along with the needle 318. As such, the tip portion 316 of the needle 318 is not exposed to the user during withdrawal of the needle 318 from a patient. In some embodiments, the needle hub 322 may be provided with a thumb grip 324 for enabling a user to grip the cannula 300 during use.

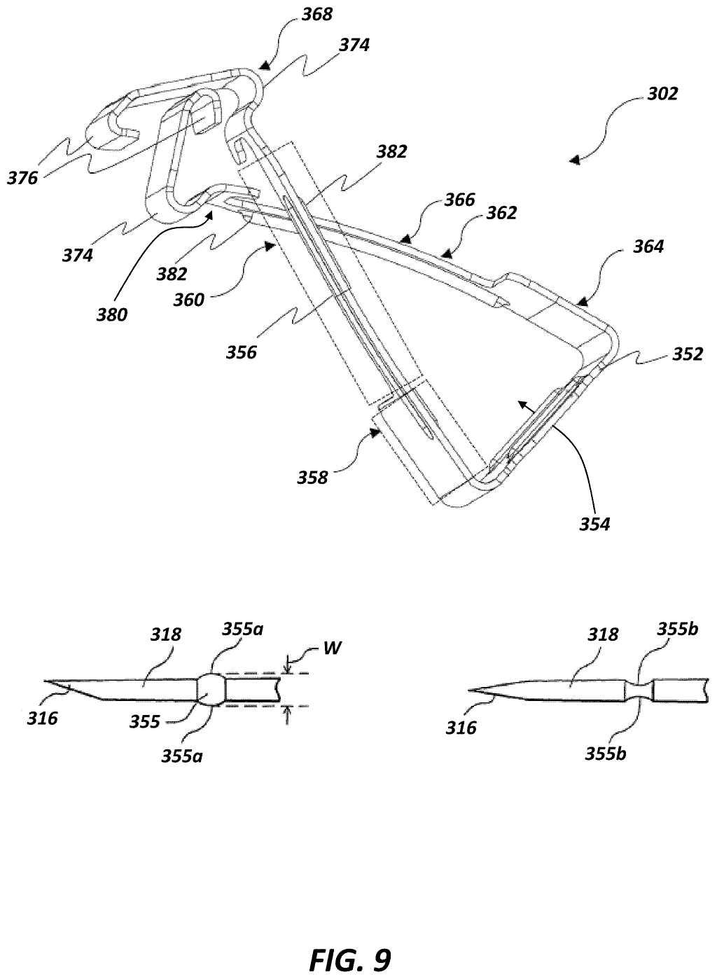

FIG. 9 is a magnified perspective of the safety clip 302. The safety clip 302 is configured to block entry of the needle 318 into the body member 308 of the catheter assembly 302 and retain the tip portion 316 of the needle 318 therein when the needle 318 is withdrawn from the body member 308. The safety clip 302 includes a bracket 352 defining an opening 354 for receiving the needle 318. The opening 354 may be sized based on the diameter of the needle 318 to be employed in the cannula 300. The opening 354 allows the needle to move proximally through the safety clip 302 until the opening 354 reaches one or more bumps, grooves, slots, bulges, crimps, flattened portions, and/or protrusions near the distal end of the needle 318 that are larger than the opening 354, so that the safety clip 302 cannot slide off the distal end of needle 318. For example, the needle 318 can define a crimp 355 near its distal tip 316. The crimp 355 in the needle 318 can include a pair of generally opposed, outwardly extending bulges 355a and a pair of generally opposed, inwardly extending depressions 355b, which are disposed generally orthogonally with respect to the bulges 355a. The bulges 355a define the crimp 355 having a width, W, which is small enough to facilitate movement of the needle 318 within the cannula 300, but large enough to prevent passage of the distal end of the needle 318 through the opening 354.

A first resilient arm 356 extends from one end of the bracket 352 and has a first section 358 and a second section 360, and the dimensions of the first section 358 may be larger than the dimensions of the second section 360 in some embodiments. As an example, the first section 358 may be wider that the second section 360. Such a construction of the safety clip 302 renders effortless and inexpensive manufacturing of the safety clip 302, while ensuring sufficient spring force or biasing spring force requirements. The first section 358 may further conform to the dimensions of the bracket 352 and thus ensure uniformity in construction, which retains the structural rigidity of the safety clip 302 when subjected to deformation.

A second resilient arm 362 extends from another end of the bracket 352 and has a first section 364 and a second section 366, and the dimensions of the first section 364 may be larger than the second section 366 in some embodiments. As an example, the first section 364 may be wider than the second section 366. Such a construction of the safety clip 302 renders effortless and inexpensive manufacturing of the safety clip 302, while ensuring sufficient spring force or biasing force requirements. The first section 364 may further conform to the dimensions of the bracket 352 and thus ensure uniformity in construction, which retains the structural rigidity of the safety clip 302 when subjected to deformation.

A connection portion 368 is also included, preferably at the second section 360. The connection portion 368 is configured to engage with at least one interlocking flange 314 configured on an inner surface of the body portion 306 and the needle 318. The connection portion 368 is configured to engage with the interlocking flange 314 and a body 370 of the needle 318 for puncturing the vein of the subject. The connection portion 368 further disengages from the interlocking flange 314 and the body 370 of the needle 318 when the needle 318 is withdrawn from the proximal end 372 of the body member 308.

The connection portion 368 includes a curved protrusion 374 at a fore end and a curved lip 376 at an aft end. The curved protrusion 374 is configured to engage with the interlocking flange 314 of the body portion 306, so that the safety clip 302 is held within the body portion 306. The curved lip 376 provided at the aft end extends inwardly toward the bracket 352 and is configured to engage the needle 318.

The curved protrusion 374 engages with the interlocking flange 314 due to biasing of the first and second resilient arms 356, 362. The first and second resilient arms 356, 362 are biased or flexed due to insertion of the needle 318 into the body member 308. Insertion or presence of the needle 318 into the needle guard assembly 304 urges the first and second resilient arms 356, 362 to flex away from each other, thereby engaging the curved protrusion 374 to engage with the interlocking flange 314. At the same time, the curved lip 376 engages with the body 370 of the needle 318, so that the biasing force is maintained and the safety clip 302 is retained within the body portion 306.

In some embodiments, the curved protrusion 374 of the first and second arms 356, 362 are outwardly extending protrusions configured for engaging the interlocking flange 314 in the body portion 306, which are inwardly protruding. Accordingly, the construction or shape or configuration of the curved protrusion 374 may be selected based on the construction of the interlocking flanges 314, so that interlocking therebetween is ensured when the needle 318 is present in the body member 308.

In another embodiment, the curved lip 376 of the first and second arms 356, 362 may comprise inwardly extending protrusions configured for engaging with the needle 318 when the needle 318 is present within the body member 308, and enclose the tip portion 316 of the needle 318 within the safety clip 302 when the needle 318 is withdrawn from the body member 308. As such, the curved lip 376 is configured to prevent entry or re-entry of the needle 318 beyond the safety clip 302, when the needle 318 is withdrawn from the body member 308. Accordingly, the construction or shape or configuration of the curved lip 376 may be selected based on the configuration of the needle 318 or the position at which the needle is inserted or withdrawn. As one non-limiting example, the curved protrusion 374 may be an inverted U-shaped member, while the curved lip 376 may be a U-shaped member.

Further, the curved protrusion 374 on each of the first and the second arms 356, 362, can include a projection 378 extending toward the bracket 352. The projection 378 along with the second section of the respective arms defines a seat portion 380. The seat portion 380 is adapted to receive and seat the tip portion 316 of the needle 318, which may ensure that the tip portion 316 of the needle 318 rests within the safety clip 302, thereby preventing misalignment of the needle 318 during withdrawal from the body member 308 or during disposal of the needle 318.

In an embodiment, the first resilient arm 356 is longer than the second resilient arm 362. Such a construction of the arms 356, 362 may be provided to ensure that sufficient biasing force is exerted onto the interlocking flanges 314 during use, thereby ensuring that the safety clip 302 is sufficiently retained within the body portion 306. Also, asymmetric lengths of the arms may ensure contact of the connection portion 368 on the body of the needle 318 about the same plane. Such an engagement may ensure uniform stress-distribution on the needle 318, thereby preventing damage during assembly. In an embodiment, the needle 318 may be assembled into the body member 308 in the body portion 306 by initially flexing the arms 356, 362 and thereafter inserting the needle 318 through the tubular sleeve.

Further, each of the bracket 352 and the first and the second resilient arms 356, 362 may be defined with at least one rib member 382. The rib member 382 reinforces the safety clip 302, which improves its overall strength. As an example, the rib member 382 may extend about the surface of the bracket 352. The rib member 382 may also extend along the second sections of the first and the second arms 356, 362, which inherently improves the strength of the second sections. The length of extension of the rib member 382 may be configured based on the strength or rigidity requirements of the safety clip 302. In an embodiment, the rib member 382 may be formed on the bracket 352 and/or the arms via conventional manufacturing techniques such as punching and the like.

In an embodiment, the rib member 382 may be provided on the bracket 352 as a reinforcement, in order to prevent damage to the bracket 352 via contact of a protuberance on the needle 318 during removal of the needle 318. Such a construction ensures that the needle 318 is retained within the safety clip 302 upon withdrawal. In some embodiments, the rib member 382 may be made of a metallic material, a plastic material, a composite material, or any other material which serves the purpose of providing reinforcement to the safety clip 302.

In an embodiment, the bracket 352 and the first and the second arms 356, 362 may be made of metallic material or any other material which serves the purpose of ensuring interlocking with the body portion 306 when the needle 318 is present within the body member 308, and encloses the tip portion 316 within when the needle 318 is withdrawn from the body member 308.

Additionally, once the tip portion 316 of the needle 318 is withdrawn from the proximal end 372, particularly, beyond the connection portion 368 of the arms 356, 362 of the safety clip 302, the biasing force acting on the arms 356, 362 due to contact with the needle 318 ceases. As such, the arms retract to an unbiased configuration, during which the curved lip 376 completely closes the path for the tip portion 316 to move forward and beyond the safety clip 302. Thus, the safety clip 302 retains the needle 318 upon withdrawal from the body member 308. In this scenario, the safety clip 302 may be withdrawn from the body portion 306 along with the needle 318 via the needle hub 322.

The tip portion 316 rests on the seat portion 380 configured on the safety pin 302, and thus the alignment of the needle 318 is maintained, irrespective of movement of the needle 318 during withdrawal. Therefore, exposure of the tip portion 316 of the needle 318 is prevented and the likelihood of needle prick injuries reduced. In embodiments, the tip portion 316 may selectively rest on any of the seat portion 380 configured on the first and second arms 356, 362.

The disclosed cannula devices may prevent the tip of the needle from contacting a user after withdrawing the needle. For instance, the disclosed needle safety components provide a safety mechanism in the form of one or more safety release components that shield the tip of the needle when the needle is withdraw after piercing a blood vessel of a patient.

The disclosed locking elements, which can include solid spherical balls in some examples, provide selective disengagement of a catheter assembly from a needle guard assembly upon proximal retraction of the needle into the needle guard assembly, thereby preventing exposure of needle tip and needle prick injuries commonly associated therewith.

The disclosed locking elements comprising solid spherical balls enable the easy removal of the needle by unlocking the locking engagement of the catheter assembly and needle guard assembly.

Embodiments described herein may also be configured to have additional or alternative needle release and retraction mechanisms. For example, embodiments of the disclosed cannulas can include a coiled spring biased to retract the needle proximally, such that the needle is automatically retracted and enclosed within the needle guard assembly after and before its deployment. Embodiments may also feature a push-button mechanism configured to facilitate release and retraction of the needle, and/or to facilitate coupling and uncoupling of the catheter assembly with the needle guard assembly. Embodiments may include a manually operable push-button protruding from, or accessible at, a surface of a cannula, extending outwardly from the needle guard assembly in some examples. Actuation of the push-button may be necessary to overcome a spring force biasing the needle in the proximal direction, within the needle guard assembly. Outward displacement of the push-button may allow the internal components of the needle guard assembly, e.g., the needle hub, to move distally in unison with the needle attached thereto. Examples of spring-biased, push-button activated cannula configurations compatible with one or more of the disclosed embodiments are described in U.S. Pat. No. 4,747,831, the entire contents of which are incorporated by reference herein.

While aspects of the present invention have been particularly shown and described with reference to the embodiments above, it will be understood by those skilled in the art that various additional embodiments may be contemplated by modification of the disclosed device without departing from the scope of what is disclosed. Such embodiments should be understood to fall within the scope of the present invention as determined based upon claims and any equivalents thereof.

1. An intravenous cannula device comprising:
a catheter assembly comprising a catheter hub coupled with a catheter body member having a distal end connected to a proximal end of a catheter tube, wherein an inner surface of a proximal portion of the catheter body member comprises an annular groove; and
a needle guard assembly configured to couple with a proximal end of the catheter assembly, the needle guard assembly comprising:
an elongated tubular member;
a needle hub comprising a needle holder disposed within the elongated tubular member, wherein a distal end of the needle holder is connected with a needle; and
a safety release component fixed to a distal end of the elongated tubular member, wherein the safety release component comprises a body and one or more moveable locking elements configured to releasably couple with the catheter body member, each of the moveable locking elements sized to fit within one of one or more complementary grooves defined by an outer surface of the body; and
wherein each of the one or more moveable locking elements is configured to engage with the annular groove of the catheter body member by moving radially outward from one of the complementary grooves, thereby coupling the elongated tubular member of the needle guard assembly with the catheter assembly when the needle is passed through the safety release component pursuant to puncturing a blood vessel of a patient.

2. The intravenous cannula device of claim 1, wherein the body of the safety release component further comprises a longitudinal bore through which the needle is passed.

3. The intravenous cannula device of claim 1, wherein the one or more moveable locking elements are spherical.

4. The intravenous cannula device of claim 3, wherein the one or more moveable locking elements are made of stainless steel.

5. The intravenous cannula device of claim 1, wherein the safety release component comprises a circular base portion fixed to a distal end of the elongated tubular member.

6. The intravenous cannula device of claim 5, wherein the safety release component comprises a tubular portion extending from the circular base portion and defining an axial bore configured to accommodate passage of the needle therethrough.

7. The intravenous cannula device of claim 6, wherein each of the one of one or more complementary grooves extends toward the axial bore of the tubular portion of the safety release component.

8. The intravenous cannula device of claim 7, wherein each of the one or more moveable locking elements is configured to settle within one of one or more complementary grooves after withdrawal of the needle from the longitudinal bore.

9. An intravenous cannula device comprising:
a catheter hub coupled with a catheter body member, the catheter body member having a proximal end, a distal end, and a coaxial recess extending from the proximal end to the distal end;
a needle guard assembly configured to couple with the proximal end of the catheter body member, the needle guard assembly comprising:
an elongated tubular member;
a needle hub comprising a needle holder disposed within the elongated tubular member, wherein a distal end of the needle holder is connected with the needle; and
a safety release component fixed to a distal end of the elongated tubular member, wherein the safety release component comprises a body and one or more moveable locking elements configured to releasably couple with the catheter body member,
wherein the one or more moveable locking elements are configured to engage with the proximal end of the catheter body member, thereby coupling the elongated tubular member of the needle guard assembly with the catheter body member when the needle is passed through the safety release component pursuant to puncturing a blood vessel of a patient.

10. The intravenous cannula device of claim 9, wherein the catheter body member comprises a Y-shaped body comprising a tangential segment extending away from the coaxial recess between the proximal and distal ends of the catheter body member.

11. A safety intravenous cannula, comprising:
a catheter assembly comprising a catheter hub, the catheter hub having a distal end connected to a proximal end of a catheter tube, and a body member adapted to accommodate the catheter hub at a distal end of the body member;
an elongated tubular member disposed at a proximal end of the catheter assembly;
a needle hub comprising a needle holder disposed inside the elongated tubular member, wherein a distal end of the needle holder is connected with a needle; and
a safety device fixedly connected to a distal end of the elongated tubular member and releasably connected to the body member of the catheter assembly.

12. The safety intravenous cannula of claim 11, wherein the body member is provided with an annular groove at an inner surface of the body member and the safety device comprises one or more locking elements.

13. The safety intravenous cannula of claim 12, wherein the one or more locking elements of the safety device are adapted to engage with the annular groove of the body member, thereby forming a locking engagement and a tight fit relationship between the elongated tubular member and the catheter assembly when the needle is passed through the safety device for puncturing a vein of a patient.

14. The safety intravenous cannula of claim 13, wherein when the needle is retracted from the catheter assembly after puncturing the vein of the patient, the body member disengages from the elongated tubular member by disengaging the locking elements from the annular groove.

15. The safety intravenous cannula as claimed in claim 11, wherein the safety device comprises a groove on an outer surface of the safety device to accommodate one or more locking elements.

16. The safety intravenous cannula of claim 15, wherein the one or more locking elements are solid spherical elements made of stainless steel material.

17. The safety intravenous cannula of claim 16, wherein the groove of the safety device has a diameter more than or equal to a diameter of the solid spherical elements.

18. The safety intravenous cannula of claim 17, wherein a first end of the safety device has a circular base portion, the circular base portion defining a central hole and fixedly connected with the distal end of the elongated tubular member.

19. The safety intravenous cannula of claim 18, wherein the safety device comprises a tubular portion extending from the circular base portion and includes an axial bore forming the passage for the needle from the central hole.

20. The safety intravenous cannula of claim 19, wherein the groove provided on the outer surface of the tubular portion of the safety device extends towards the axial bore of the safety device.

21. The safety intravenous cannula of claim 20, wherein the solid spherical elements of the safety device are adapted to move outwardly to engage with the annular groove of the body member, thereby forming the locking engagement and the tight fit relationship between the elongated tubular member and the catheter assembly when the needle is passed through the safety device for puncturing the vein of the patient.
